(19) United States
(12) Patent Application Publication
(10) Pub. No.: US 2004/0162570 A1
(43) Pub. Date: Aug. 19, 2004

(54) Coronary inflow occlusion and anastomotic assist device

(76) Inventors: Hitendu H. Dave, Gujarat (IN); Jeffrey B. Jump, Prangins (CH); Leo James Lichte, Riverside, CA (US); Marko Turina, Zurich (CH); Gregor Zund, Zurich (CH)

Correspondence Address: KNOBBE MARTENS OLSON & BEAR LLP, 2040 MAIN STREET, FOURTEENTH FLOOR, IRVINE, CA, 92614, US

(21) Appl. No.: 10/350,915

(22) Filed: Jan. 22, 2003

Related U.S. Application Data
(60) Provisional application No. 60/351,356, filed on Jan. 22, 2002.

(51) Int. Cl. A61B 17/08

(52) U.S. Cl. 606/158

(57) ABSTRACT

A surgical assist device used to constrict blood perfusion during surgeries is provided. The device utilizes one or more compression members to apply a controlled pressure to an open artery to stop blood perfusion at the surgical site. The compression members can be deployed and retracted manually, automatically, or remotely. The applied pressure can also be remotely monitored and controlled. The device can be used in conjunction with existing suction based coronary stabilization devices or used as a stand-along unit. The device can be used to apply controlled pressure to the arteries to constrict blood perfusion with significantly less invasion into the surgical area.

### RELATED APPLICATIONS

This application claims the benefit of U.S. Provisional Patent Application No. 60/351,356, filed on Jan. 22, 2002, the entirety of which is hereby incorporated by reference.

### BACKGROUND OF THE INVENTION

1. Field of the Invention

This invention relates to methods and apparatus for performing surgical procedures and, more particularly, to a method and apparatus for constricting perfusion of blood at surgical sites.

2. Description of the Related Art

In recent years, great improvements have been made in the field of performing open heart surgeries. Advances in surgical technique and equipment allow surgeons to perform open heart surgical procedures such as coronary bypass anastomosis while the patient's heart is beating. This relatively new surgical technique eliminates the need of stopping the patient's heart and placing the patient on a Cardiopulmonary Bypass System, commonly known as a heart-lung machine, during open heart surgery, which greatly reduces health risks and complications associated with conventional open heart surgeries.

Beating heart surgeries are made possible in large part by the development of local cardiac stabilization devices. These devices are designed to stabilize localized regions of the heart so as to facilitate performance of procedures such as microscopic graft to coronary anastomosis. Generally, a stabilizer uses compression or suction to steady the heart's movement in a one- to two-centimeter section while the surgeon is performing a procedure on the section.

An exemplary conventional cardiac stabilizer is vacuum based and uses a series of suction pads to stabilize a small, localized region of a beating heart. The suction pads may be attached to each other to form an elongated suction limb. One or more suction limbs can be placed on the patient's heart adjacent to the surgical site such that each suction pad is in contact with the surface of the heart. When vacuum is applied, the suction pads stretch the surrounding surface tissue, thus imparting a certain amount of rigidity to the tissue, which in turn provides a stabilized localized region for the surgeon to perform the procedure. Some commonly known vacuum based stabilizers are OCTOPUS I, II, and III brand devices manufactured by Medtronics of Minneapolis, Minn. Other stabilizers are manufactured by companies such as Guidant of Temecula, Calif. and Genzyme of Cambridge, Mass. Stabilization of the heart during open heart surgery with these various stabilizers has been relatively successful.

However, during beating heart coronary surgeries, the coronary arteries being operated on, if not constricted, will continue to perfuse blood throughout the procedure. Blood perfused from the arteries can block the surgeon's field of vision at the coronary arteriortomy site and make the procedure difficult to perform. To address this problem, a number of coronary inflow occlusion methods have been developed to constrict the perfusion of blood at the surgical site. For example, prolene slings have been developed to be placed around the artery to inhibit blood inflow during beating heart anastomosis procedures. However, it is widely recognized that the amount of force applied by the prolene sling cannot be continuously controlled. Thus, concerns remain about the safety of this device, particularly the potential vascular trauma it is likely to cause. In fact, some studies have attributed follow up native coronary stenosis to the past sites of prolene slinging. Elastic “silastic” slings that circumvent the coronary being grafted raise similar safety concerns as the pressure being applied by the elastic slings also cannot be controlled. The slings are applied to the arteries at various degrees of pressure, depending on the force exerted by the person applying the sling. Additionally, application of the sling to the artery requires manual dexterity and technique that some less experienced surgeons may not have yet perfected. It is also difficult to construct and maintain the elastic sling on the coronary branches at the inferior cardiac surface.

Other aortic cross clamping devices such as sharp tipped micro bulldogs (clamps) have also been used to inhibit coronary inflow. However, the sharp tipped clamps can cause trauma not only to the coronary artery but also the myocardium. The conventional clamps also have a high profile and thus can hamper the surgical procedure especially on the inferior cardiac surface. Intracoronary shunts are effective in inhibiting the blood inflow, however they can be difficult to place and potentially can cause vascular trauma during placement.

Hence, from the foregoing, it will be appreciated that there is a need for a safer and less invasive method of inhibiting coronary inflow during open heart surgical procedures. To this end, there is a particular need for an apparatus that reduces trauma to the arteries and surrounding tissues while inhibiting blood perfusion from the arteries during beating heart coronary bypass procedures. There is also a particular need for a surgical assist device that is configured to apply a controlled amount of pressure to arteries to block the flow of blood during coronary bypass anastomosis procedures.

### SUMMARY OF THE INVENTION

In one aspect, the preferred embodiments of the present invention provides a device for occluding blood flow in arteries during surgical procedures. The device comprises a frame adapted to be positioned adjacent an artery, at least one occlusion member movably mounted to the frame, wherein the occlusion member can be moved with respect to the frame so as to engage the artery to thereby inhibit the flow of blood in the artery. The device further comprises an actuation mechanism coupled to the at least one occlusion member so as to controllably move the at least one occlusion member into engagement with the artery such that the amount of force exerted on the artery can be controlled during occlusion of blood flow in the artery.

In one embodiment, the frame is substantially rectangular and is sized so as to engage with a vacuum cardiac stabilizer. Moreover, at least one occlusion member is preferably mounted on at least the first end of the frame. In another embodiment, the actuation mechanism is adapted to permit a treating medical professional to select and maintain a substantially constant amount of force on the artery by the at least one occlusion member during the surgical procedure. Preferably, the at least one occlusion member comprises a pair of occlusion members that are adapted to engage with two ends of a severed artery. In one embodiment, the at least one occlusion member comprises a roller that is pivotally attached to the frame such that the roller can be moved in an arc to thereby engage the artery. In another embodiment, the roller has a circumference in the range of approximately 0.5 inch to 0.850 inch.

In another embodiment, the at least one occlusion member includes an arm that is pivotally attached to the frame, wherein the arm defines a roller end to which the roller is mounted to an actuation end such that movement of the actuation end results in the corresponding movement of the roller end. The actuation mechanism can comprise a controllable actuator that applies a controlled amount of force on the actuation end of the arm. Preferably, the actuation mechanism is adapted to maintain a constant force on the actuation end of the arm. In other embodiments, the actuation mechanism includes a feedback device that provides a visual indication of the amount of force being applied to the actuation end of the arm to permit a treating medical professional to select and maintain the amount of force being exerted on the artery by the roller.

In another aspect, the preferred embodiments of the present invention comprises an assembly for performing surgery on arteries adjacent the heart of a patient. The assembly comprises an artery occlusion assembly that includes a frame, at least one controllable artery occlusion member mounted on the frame and an associated actuator, wherein the associated actuator permits the application and maintenance of a selected amount of pressure on the artery by the at least one controllable artery occlusion member so as to at least partially occlude blood flow in the artery. The assembly further comprises a fluid supply system mounted adjacent the frame so as to provide a fluid stream to a location adjacent the artery to thereby facilitate removal of blood from the location adjacent the artery.

In one embodiment, the at least one controllable artery occlusion member comprises a first and a second member mounted on opposed sides of a substantially rectangular frame. The at least one controllable artery occlusion member is pivotally mounted to the frame so as to continuously pivot between an occluding position and a release position. Preferably, the actuator assembly is adapted to apply force to the at least one controllable artery occlusion member so as to induce the at least one controllable artery occlusion member to pivot into a first position and to substantially maintain the controllable artery occlusion member in the first position. Moreover, the actuator can also include a feedback device that provides a visual indication of the amount of force being applied to the controllable artery occlusion member to permit a treating medical professional to select and maintain the amount of force being exerted on the artery.

In yet another aspect, the preferred embodiments of the present invention provide a surgical assist device for constricting blood perfusion from an opening in an artery. The device comprises at least one compression member, wherein the compression member can be deployed to contact the artery and apply a gradually increasing pressure to the artery until a first pressure is reached. Preferably, the first pressure is substantially the least amount of pressure required to constrict the artery so as to block blood from flowing out of the opening in the artery. Moreover, the device also comprises a locking mechanism that locks the compression member in position when the first pressure is reached so that the compression member continues to apply the first pressure to the artery.

Advantageously, the preferred embodiments of the present invention provide a surgical assist device that is designed to aid surgeons and other medical professionals in maintaining a clear surgical field at the surgical area, such as the anastomotic site, by blocking the flow of blood through the opened coronary arteries. The device accomplishes this with substantially less invasion into the surgical area as compared with the conventional artery clamping devices. Moreover, the device is designed to constrict blood flow with very little intervention from the surgeon other than to initially activate the device to perform the task.

### BRIEF DESCRIPTION OF THE DRAWINGS

FIG. 1 is a partial schematic illustration of a surgical assist device of one preferred embodiment of the present invention;

FIGS. 2A and 2B are partial schematic illustrations of a surgical assist device of another preferred embodiment of the present invention;

FIG. 3 is a schematic illustration of the surgical assist device of FIG. 1 used in conjunction with a conventional cardiac stabilizer during beating heart coronary surgeries;

FIG. 4 is partial schematic illustration of a surgical assist device of another embodiment of the present invention used in conjunction with a cardiac stabilizer.

### DETAILED DESCRIPTION OF THE PREFERRED EMBODIMENT

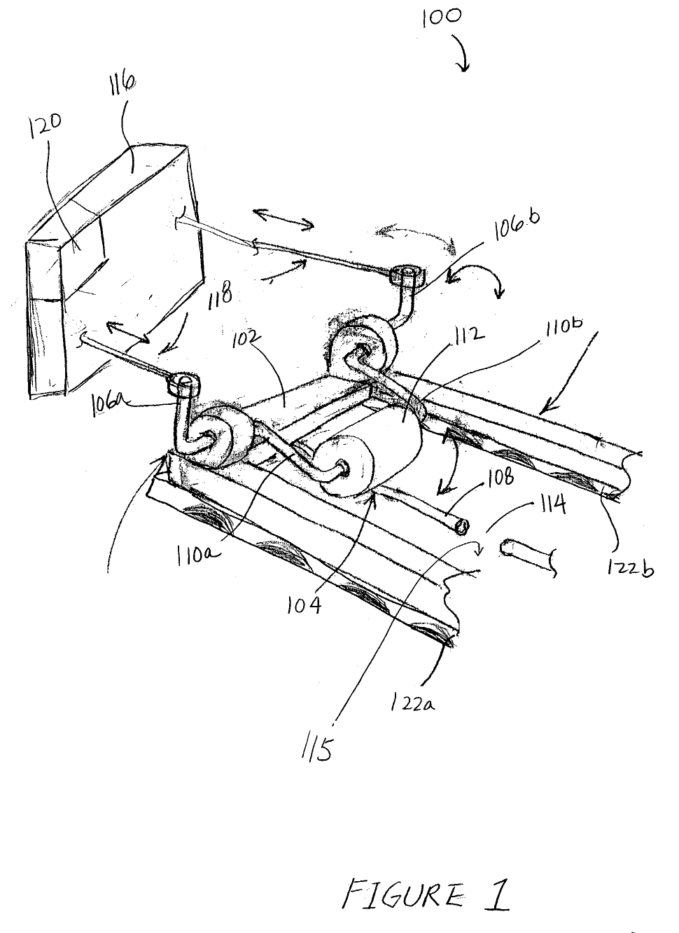

Reference will now be made to the drawings wherein like numerals refer to like parts throughout. FIG. 1 provides a partial schematic illustration of a surgical assist device 100 of one preferred embodiment of the present invention. As shown in FIG. 1, the surgical assist device 100 generally comprises a platform 102, one or more compression members 104 pivotally mounted on the platform 102, and a plurality of levers 106a, 106b operatively interconnected to the compression member 104. As will be described in greater detail below, the compression member 104 is adapted to apply pressure to an artery 108 in a gradual and controlled manner to constrict blood flow during surgeries so as to reduce vascular trauma.

In one embodiment, the compression member 104 comprises two pivoting compression legs 110a, 110b fitted with one or more horizontally placed cylindrical rollers 112. The roller 112 can be raised or lowered by pivoting the compression legs 110a, 110b. In one embodiment, the compression legs 110a, 110b are pivoted by rotating the levers 106a, 106b that are operatively interconnected to the legs 110a, 110b. When the compression legs 110a, 110b are in a deployed position as shown in FIG. 1, the roller 112 extends toward the artery 108, contacts the eipcardial surface of the artery 108, and begins to compress the artery to inhibit blood from perfusing from an opening 114 in the artery 108 at a surgical site 115. When sufficient compression has been achieved to stop blood perfusion, the compression member 104 can be locked in place so that it maintains the position, thereby applying a continued and fixed pressure to the artery. In one embodiment, the compression member 104 can be locked in position using a locking mechanism such as a ratchet mechanism, a frictional engagement mechanism, or any other known mechanisms.

When the surgeon wants to reduce the pressure, the compression member can be “unlocked” and retracted to its original position or a new position that applies less pressure to the artery. The ability to control or “lock in” the amount of pressure applied to an artery reduces the occurrence of vascular trauma resulting from over-compression. Moreover, the large contact surface provided by the roller 112 distributes the applied pressure over a larger cross-sectional area of the artery, which further reduces vascular trauma. In one embodiment, the roller 112 has a width of about 0.25 inch, a radius of about 0.125 inch. Moreover, the contact surface area is at least about 0.10 inch. Moreover, the hardness of the roller 112 can also be modified to vary the amount of pressure applied to the arteries. The roller 112 can be made of stainless steel, hard plastic, soft plastic, and the like.

The compression member 104 can be manually extended or retracted by moving the levers 106a, 106b. Alternatively, the compression member 104 can also be remotely activated by a remote activation device 116, using wires or conduits 118 that allow for the use of vacuum, gas/fluid pressure, electromechanical power such as a small motor. Furthermore, the compression member 104 can also be remotely controlled by mechanical devices or by radio or light frequencies such as ultrasonic, subsonic, infrared, or the like. One advantage derived from the ability to operate the device 100 remotely is that it reduces the amount of space taken up by the device 100 if it were controlled at the site. Furthermore, the ability to vary the amount of coronary compression from a remote site can be very useful in myocardial ischaemic preconditioning. The same feature can be used to an advantage to fill up the coronary at various stages of constructing the coronary anastomosis.

In one embodiment, the device 100 is provided with a built-in pressure monitor 120 that allows the surgeon to monitor the amount of pressure being applied to the artery by the compression member. When the proper pressure has been achieved, the compression member 104 is automatically locked in place using a known mechanism in a manner such that the compression member maintains the position and thereby the pressure on the artery. When the surgeon wants to reduce or release the pressure on the artery, the locking mechanism is deactivated, and the arm or lever can be retracted to its original position.

As FIG. 1 further shows, the compression member 104 is mounted on the platform 102, whereby the platform 102 can be placed directly over the surgical site 115 or affixed to a cardiac stabilizer already in place adjacent the surgical site. In one embodiment, the platform 104 comprises a rectangular frame that can be attached to a conventional cardiac stabilization device such as the OCTOPUS I, II, or III brand stabilizers or stabilizers disclosed in U.S. Pat. Nos. 5,836,311, 5,927,284, 6,015,378, 6,328,688, all entitled “METHOD AND APPARATUS FOR TEMPORARILY IMMOBILIZING A LOCAL AREA OF TISSUE”, each of which is hereby incorporated by reference in its entirety.

As shown in FIG. 1, the platform 102 can be attached to parallel suction limbs 122a, 122b that are typically part of a vacuum based stabilizer. The platform 104 can be attached to the suction limbs 122a, 122b via a number of different attachment methods. For example, temporary two-sided adhesive tapes can be used to adhere a lower surface of the platform to an upper surface of the suction limb. Other attachment methods include using a “snap-on” or “clip-on” configuration that would allow the platform to be held in place temporarily on the suction limbs 122a, 122b or other sections of the stabilizer. Advantageously, the platform 102 can be detached from the stabilizer after the completion of the surgery.

In another embodiment, the platform 102 can be permanently attached to the stabilizer 122a, 122b and can be disposed or reused along with the stabilizer when the surgery is complete. Furthermore, the compression member 104 can be positioned anywhere between the suction limbs 122a, 122b of the stabilizer so as to be able to specifically target the location of the compression, which is often times precisely on the epicardial surface of the coronary artery. This ability makes up for the tortuously running coronary arteries which may not necessarily be in the middle of the suction limbs.

Furthermore, the surgical assist device 100 of one preferred embodiment of the present invention has two compression members 104 spaced apart in a manner such that each compression member is used to inhibit blood perfusion from different segments of the artery, preferably segments of the artery on opposite sides of the opening in the artery. Advantageously, the artery compression provided by the preferred surgical device 100 proceeds gradually until the point when coronary inflow is inhibited, which is an effective indicator of when to stop increasing the pressure so as to not over-compress. In operation, the compression member can be extended over the epicardial surface of the coronary artery until a certain pressure is achieved wherein coronary inflow is stopped. Thus, the amount of pressure exerted on the arteries can be consistently applied and is not dependent on the particular surgeon.

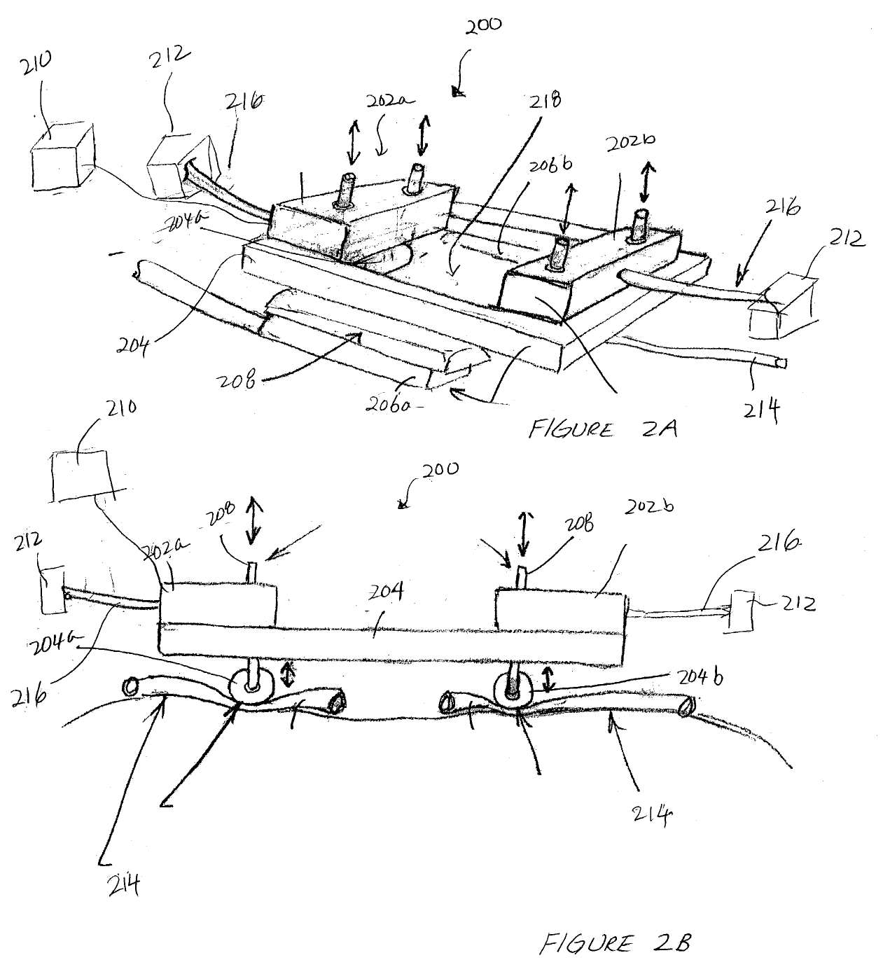

FIGS. 2A and 2B are schematic illustrations of a surgical assist device 200 of another embodiment of the present invention. As shown in FIG. 2A, the surgical device 200 incorporates a plurality of compression members 202a, 202b mounted on a platform 204 that is removably attached to a plurality of suction limbs 206a, 206b. A plurality of device mounting adapters 208 such as two-sided tapes can be used to mount the platform 204 onto the suction limbs 206a, 206b. FIG. 2B provides a schematic side view of the device 200 showing that the compression members 202a, 202b have contact surfaces comprised of cylindrical rollers 204a, 204b, however it can be appreciated that the contact surface may be comprised of a number of other surfaces other than cylindrical rollers. In this embodiment, the compression members 202a, 202b are configured to a move up and down, in a piston-like manner, over an artery 214 to release and apply pressure to the artery 214. Preferably, a plurality of roller axle and levers 208 are connected to the compression member 202a, 202b so that when the levers 208 are activated, the compression member 202a, 202b moves in an up and down manner. Similar to the embodiment shown above in FIG. 1, the levers can be manually or automatically activated. Furthermore, the amount of pressure applied by the compression members 202a, 202b can be remotely monitored and controlled by a remote activation device 210.

As FIGS. 2A and 2B show, the surgical device 200 further incorporates a flexible conduit delivery system which provides for the delivery of fluid, gas, or a combination of fluid and gas through a conduit 216 positioned between the existing two suction limbs 206a, 206b of the stabilizer. As shown in FIG. 2A, a plurality of conduits 216 extend from an external source 212 through the device platform 204 to deliver fluid or gas to the surgical site 218. The conduit 216 preferably has single or multiple small holes that can deliver fluid, gas or a combination of both at a precise point in between the suction limbs. The exact position of the nozzle or delivery end of the conduit can be located precisely in the area preferred by the user. The positioning of the conduit can be obtained either by adjusting the conduit manually to the desired location or remotely through the use of levers, wires, or electro-mechanically with motors, and using a remote control that activates the positioning features through use of radio or light frequencies. The delivery system may be used to deliver liquids such as saline or gas such as carbon dioxide, or a combination thereof, which help in clearing away blood that may sometimes leak at the surgical site. In particularly, positioning the nozzle at the crucial stages of the anastomosis construction helps to open up the collapsed graft as well as fill up the native coronary, and thus aids in performing a secured anastomosis.

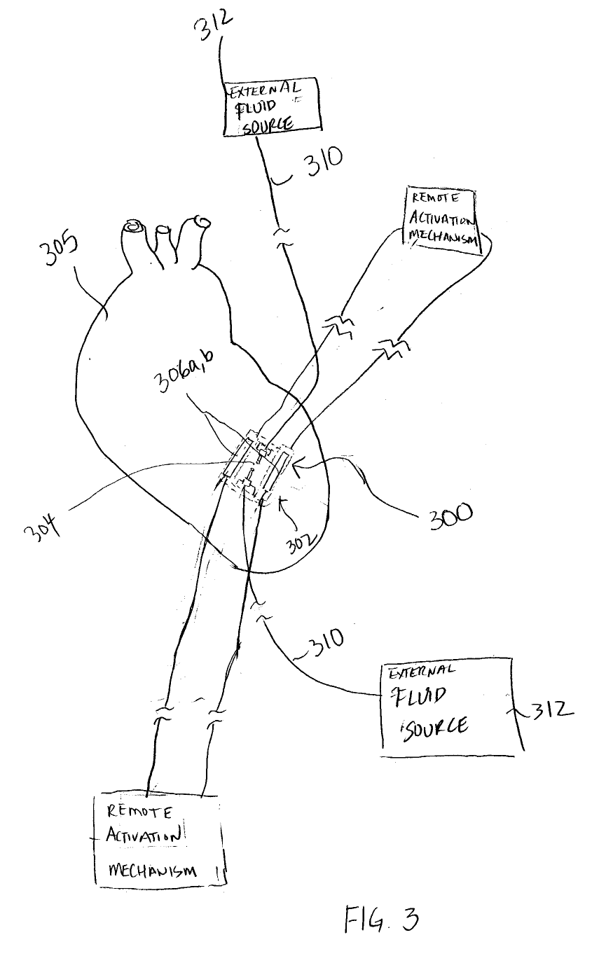

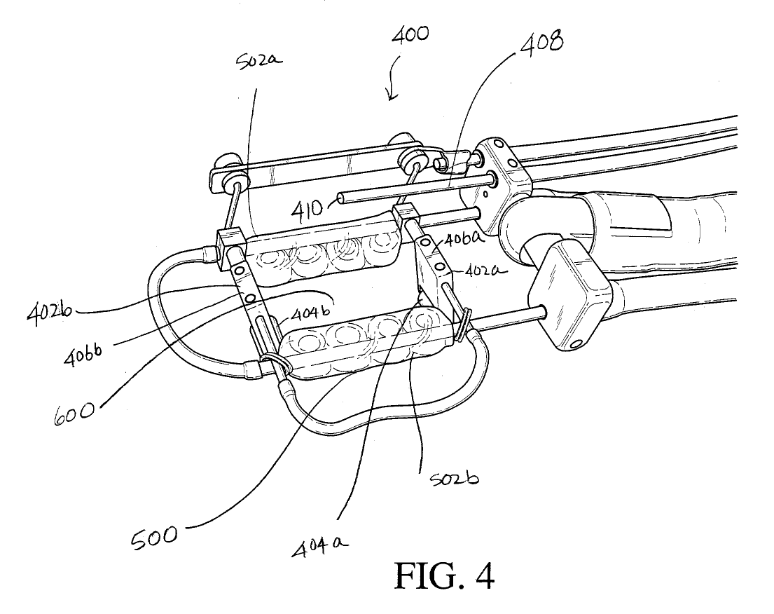

FIGS. 3 is a schematic illustration of a preferred manner in which a surgical assist device 300 of one preferred embodiment can be used in conjunction with a conventional coronary stabilizing device 302 during a beating heart coronary bypass surgery. As shown in FIG. 4, the stabilizing device 302 is positioned adjacent to a surgical site 304 on a heart 305. The stabilizer 302 is preferably removably affixed to an outer surface of the heart 305 via suction limbs 306a, 306b that are attached via vacuum to tissues surrounding the surgical site 304. The surgical assist device 300 is positioned on an upper surface of the stabilizer 302. The device 300 can be attached to the stabilizer 302 by a number of different methods. For example, the device 300 can be attached to the upper surface of the stabilizer via temporary two-sided adhesive tapes, “snap-on” or “clip-on” configuration, which allows the device to be held in place temporarily. The device can be detached from the stabilizer after the completion of the procedure for a particular artery. In another embodiment, the device 300 can be permanently attached to the stabilizer 302 so that they can be disposed of together. As FIG. 3 further shows, a plurality of fluid delivery conduits extend between an external fluid source 312 and the surgical site 304 so as to provide fluid to clear the surgical site 304 during the operation.

FIGS. 4 is a partial schematic illustration of a surgical assist device 400 of another preferred embodiment, showing the manner in which the device 400 can be used in conjunction with a conventional coronary stabilizer 500 at a surgical site 600. As FIG. 4 shows, the device 400 is mounted adjacent to two suction limbs 502a, 502b of the stabilizer 500. The device 400 comprises a plurality of occlusion members 402a, 402b, each occlusion member 402a, 402b comprising a roller 404a, 404b that is mounted on a block 406a, 406b. The block 406a, 406b can be pivoted in a known manner so as to deploy and retract the roller 404a, 404b to and from the surgical site 600 to apply compression to an artery. Moreover, the device 400 further comprises a fluid delivery tube 408 having an opening 410 position between the two suction limbs 502a, 502b. The fluid delivery tube 408 provides fluid to clear the surgical site of extraneous blood so that the medical professional can maintain a clear field of vision.

Advantageously, the surgical assist device of the preferred embodiments of the present invention can be used in conjunction with existing suction based coronary stabilization devices such as the “OCTOPUS III” brand manufactured and sold by Medtronic, Guidant or similar devices that are available in the future. Additionally, the preferred surgical device can also be used as a stand alone device or in conjunction with other medical devices other than the type of stabilization devices akin to OCTOPUS II brand. The preferred surgical device aids the surgeon in maintaining a clear surgical field at the surgical site by blocking the flow of blood through the opened coronary artery during a beating heart surgical procedure. This is accomplished with a minimum invasion into the surgical area, and with little intervention from the surgeon other than to initially activate the device to perform the task. Moreover, numerous sources of power or forces can be applied to the compression member to control the extension or retraction, including straight mechanical levers, wires, vacuum, pressure, and electromechanical power such as a miniaturized motor. As such, the device allows a gradual and consistent pressure to be applied to the arteries which reduces the risk of vascular trauma.

Although the preferred embodiments of the present invention has shown, described and pointed out the fundamental novel features of the invention as applied to the embodiments herein, it will be understood that various omissions, substitutions and changes in the form of the detail of the device illustrated may be made by those skilled in the art without departing from the spirit of the present invention. Consequently, the scope of the invention should not be limited to the foregoing description.

What is claimed is:

1. A device for occluding blood flow in arteries during surgical procedures, the device comprising:
a frame adapted to be positioned adjacent an artery;
at least one occlusion member movably mounted to the frame, wherein the occlusion member can be moved with respect to the frame so as to engage the artery to thereby inhibit the flow of blood in the artery;
an actuation mechanism coupled to the at least one occlusion member so as to controllably move the at least one occlusion member into engagement with the artery such that the amount of force exerted on the artery can be controlled during occlusion of blood flow in the artery.

2. The device of claim 1, wherein the frame is substantially rectangular and is sized so as to engage with a vacuum cardiac stabilizer.

3. The device of claim 2, wherein the at least one occlusion member is mounted on at least the first end of the frame.

4. The device of claim 1, wherein the actuation mechanism is adapted to permit a treating medical professional to select and maintain a substantially constant amount of force on the artery by the at least one occlusion member during the surgical procedure.

5. The device of claim 1, wherein the at least one occlusion member comprises a pair of occlusion members that are adapted to engage with two ends of a severed artery.

6. The device of claim 1, wherein the at least one occlusion member comprises a roller that is pivotally attached to the frame such that the roller can be moved in an arc to thereby engage the artery.

7. The device of claim 4, wherein the roller has a circumference in the range of approximately 0.5 inch to 0.85 inch.

8. The device of claim 4, wherein the at least one occlusion member includes an arm that is pivotally attached to the frame, wherein the arm defines a roller end to which the roller is mounted to an actuation end such that movement of the actuation end results in corresponding movement of the roller end.

9. The device of claim 6, wherein the actuation mechanism comprises a controllable actuator that applies a controlled amount of force on the actuation end of the arm.

10. The device of claim 7, wherein the actuation mechanism is adapted to maintain a constant force on the actuation end of the arm.

11. The device of claim 8, wherein the actuation mechanism includes a feedback device that provides a visual indication of the amount of force being applied to the actuation end of the arm to permit a treating medical professional to select and maintain the amount of force being exerted on the artery by the roller.

12. An assembly for performing surgery on arteries adjacent the heart of a patient, the assembly comprising:
an artery occlusion assembly comprising a frame, at least one controllable artery occlusion member mounted on the frame and an associated actuator, wherein the associated actuator permits the application and maintenance of a selected amount of pressure on the artery by the at least one controllable artery occlusion member so as to at least partially occlude blood flow in the artery; and
a fluid supply system mounted adjacent the frame so as to provide a fluid stream to a location adjacent the artery to thereby facilitate removal of blood from the location adjacent the artery.

13. The assembly of claim 12, wherein the frame is substantially rectangular in shape.

14. The assembly of claim 13, wherein the at least one controllable artery occlusion member comprises a first and a second member mounted on opposed sides of the substantially rectangular frame.

15. The assembly of claim 13, wherein the at least one controllably artery occlusion member is pivotally mounted to the frame so as to continuously pivot between an occluding position and a release position.

16. The assembly of claim 15, wherein the actuator assembly is adapted to apply force to the at least one controllable artery occlusion member so as to induce the at least one controllable artery occlusion member to pivot into a first position and to substantially maintain the controllable artery occlusion member in the first position.

17. The assembly of claim 16, wherein the actuator includes a feedback device that provides a visual indication of the amount of force being applied to the controllable artery occlusion member to permit a treating medical professional to select and maintain the amount of force being exerted on the artery.

18. The assembly of claim 13 wherein the at least one controllable artery occlusion member comprises a roller having a circumference in the range of approximately 0.5 inch to 1.0 inch.

19. The assembly of claim 12, further comprising a cardiac stabilizing device.

20. A surgical assist device for constricting blood perfusion from an opening in an artery, comprising:
at least one compression member, wherein the compression member can be deployed to contact the artery and apply a gradually increasing pressure to the artery until a first pressure is reached, wherein the first pressure is substantially the least amount of pressure required to constrict the artery so as to block blood from flowing out of the opening in the artery;
a locking mechanism, wherein the locking mechanism locks the compression member in position when the first pressure is reached so that the compression member continues to apply the first pressure to the artery.

21. The device of claim 20 further comprising a frame, wherein the frame is configured to support the at least one compression member, wherein the frame can be mounted to a cardiac stabilizing device.

22. The device of claim 20, wherein the at least one compression member can be deployed using an automatic actuator.

23. The device of claim 20, wherein the at least one compression member can be deployed remotely.

24. The device of claim 20, wherein the compression member can be moved to a retracted position when constriction of blood flow through the artery is no longer needed.

25. The device of claim 24, wherein the compression member can be deployed and retracted manually.

26. The device of claim 20, wherein the compression member has a contact surfaced, wherein the contact surface is adapted to contact and press against the artery, wherein the contact surface is configured so that pressure is substantially evenly applied to the artery over the entire contact surface.

27. The device of claim 20, wherein the compression member comprises a cylindrical device that is adapted to contact and press against the artery.
